(19) United States
(12) Patent Application Publication
(10) Pub. No.: US 2006/0105967 A1
(43) Pub. Date: May 18, 2006

(54) Flavone derivatives as TNFalpha inhibitors or antagonists

(75) Inventors: Li-Wei Hsu, Taichung City (TW); Su-Chen Chang, Taichung City (TW); Chen-Hsiang Shen, Xizhi City (TW); Yuan-Xiu Liao, Kaohsiung City (TW); Kuo-Sheng Chuang, Jian Township (TW)

Correspondence Address: Scully, Scott, Murphy & Presser, 400 Garden City Plaza, Garden City, NY, 11530, US

(73) Assignee: Advanced Gene Technology, Corp., Taichung City (TW)

(21) Appl. No.: 10/992,178

(22) Filed: Nov. 18, 2004

(51) Int. Cl. A61K 31/7048; A61K 31/353

(52) U.S. Cl. 514/27; 514/456

(57) ABSTRACT

The use of flavone derivatives of formula (I) 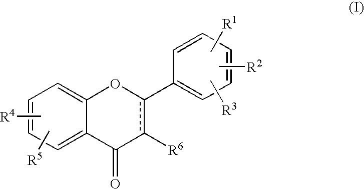
in which R¹, R², R³, R⁴ and R⁵ independently represent hydrogen, hydroxy or an ester group; R⁶ represents hydrogen, hydroxy, an ester group or an O-glycoside group such as O-rhamnose, O-glucoside, O-retinoside or O-xyloside; and [symbol] represents a single bond or a double bond; or the pharmaceutically acceptable salts thereof as TNFα antagonists or inhibitors.

### FIELD OF THE INVENTION

The present invention relates to the use of flavone derivatives as TNFα (tumor necrosis factor-α) antagonists or inhibitors.

### BACKGROUND OF THE INVENTION

Flavonoids are a group of polyphenolic compounds exhibiting a variety of important bioactivities such as anti-inflammatory, antihepatotoxic and anti-ulcer actions. They also inhibit enzymes such as aldose reductase and xanthine oxidase. They are potent antioxidants and have free radical scavenging abilities. Many have antiallergic, antiviral actions and some of them provide protection against cardiovascular mortality. They have been shown to inhibit the growth of various cancer cell lines in vitro, and reduce tumour development in the experimental animals (Narayana et al., Indian Journal of Pharmacology 2001; 33: 2-16).

Flavonoid compounds disclosed in WO 01/64701, or U.S. Pat. No. 6,706,865, has a chemical structure of formula (II) 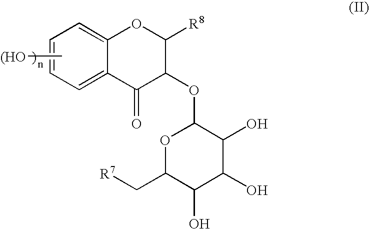
in which R⁸ is a substituted or unsubstituted phenyl group; R⁷ is a hydrogen atom or a hydroxyl group; and n is an integer of 1 to 4 and have reductase inhibitory effect, active oxygen extinguishing effect, carcinogenesis promotion inhibitory effect, anti-inflammatory effect, and so on. Astilbin is a flavanone represented by the following formula (III) 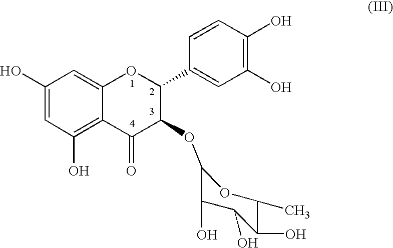
and is one of digydroflavonol glycoside isolated from root of Astilbe thunbergii Miq., which is gerbaceous perennial of saxifragaceous, as well as from the plant matter of Asmilaxylabra, Engelhardtia, Lyoniaovalifolia, Engelhardtiachrysolepos, Chloranthus glarber, Astilbe, microphylla, and so on. Astilbin has been reported to exhibit some important bioactivities such as aldose redutase inhibitory effect, active oxygen extinguishing effect, carcinogenesis promotion inhibitory effect, anti-inflammatory effect, and so on (Japanese Patent Publication Nos. 97/30984, 94/247851, and 94/256194), and therefore, astilbin is to be a very useful compound as anti-allergic drug or anticancer drug. However the anti-inflammatory mechanism has not yet been established. Of the several inflammatory mediators known to date, TNFα is one of by far the most potent and characterized cytokines, it is selected to test whether flavone derivatives inhibit the binding of TNFα to TNFα-R1 by L929 cell proliferation/cytotoxicity assay.

TNFα plays an important role in the host defense. It causes resistance to many pathogenic microorganisms and some viruses. Even if TNFα has undoubtedly a beneficial function (mainly on the systematic level), it could lead to pathological consequences. TNFα plays a significant role in the pathogenesis of septic shock, characterized by hypotension and multiple organ failure among others. TNFα is the main mediator of cachexia characterized by abnormal weight-loss of cancer patients. Often TNFα is detected in the synovial fluid of patients suffering from arthritis. There was a broad spectrum of diseases, where TNFα could play an important role. Compounds binding with TNFα may be therefore useful in the treatment of numerous pathologies in which TNFα is involved, such as rheumatoid arthritis, Crohn's disease, plaque sclerosis, septic shock, cancer or cachexia associated with an immunodeficiency.

### SUMMARY OF THE INVENTION

It has been found by the present inventor that a flavone derivative of formula (I) 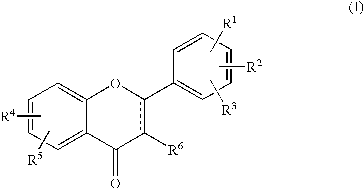
in which R¹, R², R³, R⁴ and R⁵ independently represent hydrogen, hydroxy or an ester group; R⁶ represents hydrogen, hydroxy, an ester group or an O-glycoside group such as O-rhamnose, O-glucoside, O-retinoside or O-xyloside; and [symbol] represents a single bond or a double bond; or the pharmaceutically acceptable salt thereof is useful for inhibiting the binding of TNFα to TNF-R1 or the release of TNFα and therefore may be used as TNFα antagonists or inhibitors in the treatment of numerous pathologies in which TNFα is involved, such as rheumatoid arthritis, Crohn's disease, plaque sclerosis, septic shock, cancer or cachexia associated with an immunodeficiency. It is found that Myricitrin, quercitrin and quercetin-3-D-glucoside exhibit an inhibitory activity with IC₅₀ values of 116.03, 160.77 and 95.74 μM on L929 cell proliferation/cytotoxicity assay without cell cytotoxicity. In addition, in the animal model of collagen-induced arthritis, the flavone derivatives exhibited 50% inhibitory activity. The flavone derivatives are promising sources with high TNFα inhibitor or antogonist activity.

Therefore, the first aspect of the present invention is a pharmaceutical composition for antagonizing or inhibiting TNFα in a mammal, including human, comprising an amount of a compound of formula (I) or the pharmaceutically acceptable salt thereof effective in antagonizing or inhibiting TNFα and a pharmaceutically acceptable carrier.

The second aspect of the present invention is a pharmaceutical composition for treating a disease or condition for which a TNFα antagonist or inhibitor is indicated in a mammal, including human, comprising an amount of a compound of formula (I) or the pharmaceutically acceptable salt thereof effective in antagonizing or inhibiting TNFα and a pharmaceutically acceptable carrier.

The third aspect of the present invention is a method for antagonizing or inhibiting TNFα in a mammal, including human, comprising administering to said mammal an amount of the compound of formula (I) or the pharmaceutically acceptable salt thereof effective in antagonizing or inhibiting TNFα.

The fourth aspect of the present invention is a method for treating a disease or condition for which a TNFα antagonist or inhibitor is indicated in a mammal, including human, comprising administering to said mammal an amount of the compound of formula (I) or the pharmaceutically acceptable salt thereof effective in antagonizing or inhibiting TNFα.

### BRIEF DESCRIPTIONS OF THE DRAWINGS

The accompanied drawings are to provide a further understanding of the invention, and are incorporated in and constitute a part of this specification. The drawings illustrate embodiments of the invention and, together with the description, serve to explain the principles of the invention.

FIG. 1 is a HPLC chromatogram of Chamaesyce hirta (L) Millsp. methanolic extract.

FIG. 2 shows the results of L929 cellular assay of Chamaesyce hirta (L) Millsp. methanolic extract.

FIG. 3 illustrates the isolation of quercitrin and myricitrin from Chamaesyce hirta (L) Millsp. methanolic extract.

FIG. 4 is a HPLC chromatogram of quercitrin.

FIG. 5 is a HPLC chromatogram of myricitrin.

FIG. 6 shows the results of L929 cellular assay on quercitrin.

FIG. 7 shows the results of L929 cellular assay on myricitrin.

FIG. 8 is a LC/MS chromatogram of quercitrin.

FIG. 9 is a LC/MS chromatogram of myricitrin.

FIG. 10 is the ¹H-NMR spectrum of quercitrin.

FIG. 11 is the ¹H-NMR spectrum of myricitrin.

FIG. 12 shows the results of inhibition assay on myricitrin, quercitrin and quercetin-3-D-glucoside.

FIGS. 13-1 to 13-10 show in vivo test results by using rats with collagen-induced arthritis.

### DETAILED DESCRIPTION OF THE INVENTION

The compound of formula (I) may be administered to mammals via oral, parenteral (such as subcutaneous, intravenous, intramuscular, intrasternal and infusion techniques), rectal, intranasal, topical or transdermal (e.g., through the use of a patch) routes, etc. The compound of formula (I) or the salt thereof may be administered alone or in combination with pharmaceutically acceptable carriers or diluents by any of the routes previously indicated, and such administration may be carried out in single or multiple doses. Suitable pharmaceutical carriers include solid diluents or fillers, sterile aqueous media and various non-toxic organic solvents, etc.

Experiments

1. Preparation of the Methanolic Extract of Chamaesyce hirta (L) Millsp.

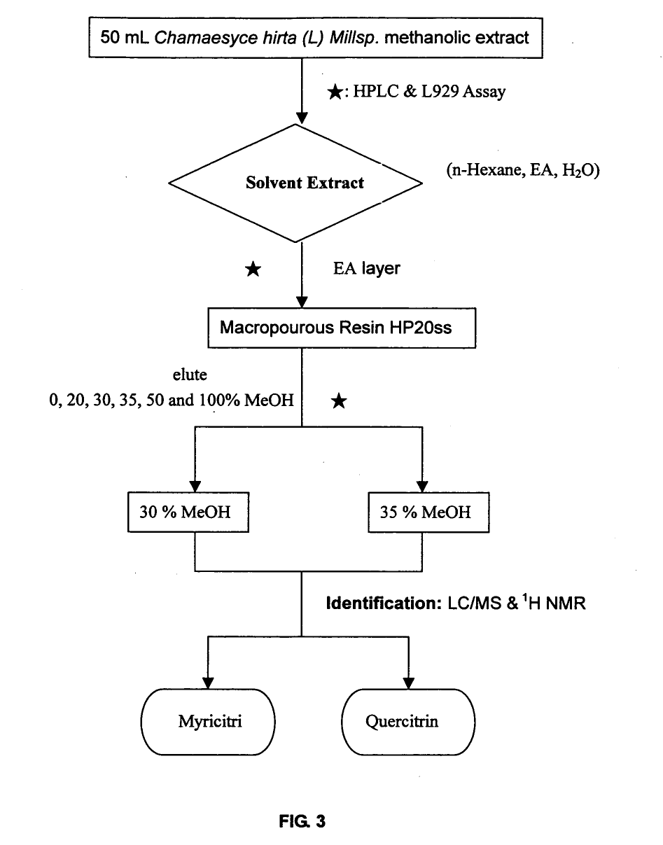

Possible TNFα inhibitor candidates were found in herbal ingredients fractionated by HPLC from herbal extract. Fifty grams of Chamaesyce hirta (L) Millsp. was washed and dried. Methanol was added to the weighed herb (10/1, v/w) to extract the herbal ingredients at room temperature for 3 days. The extract was filtered and the filtrate was concentrated under rotatory evaporator (Heidolph Laborota 4000) until the volume was reduced to about 50 mL. (FIG. 3)

2. HPLC Analysis of the Methanolic Extract Obtained From Chamaesyce hirta (L) Millsp.

Then a separation procedure was performed. One hundred μl of the concentrated filtrate of the herb extract was applied to a pre-equilibrated HPLC system (Shimadu). A TSK Gel 80™ reverse phase column (TOSOH) was used for separation. The solvent used for separation was double distilled water and absolute ethanol at 0˜100% gradient for 96 minutes at a flow rate of 0.75 mL/min.

One-minute fractions were collected and dried using SpeedVac (Savant). Each fraction was re-dissolved in 100 μl 10% ethanol for screening for TNFα inhibitors. The fractions with TNFα inhibitor activity were then further purified by HPLC until the purity was more than 95%.

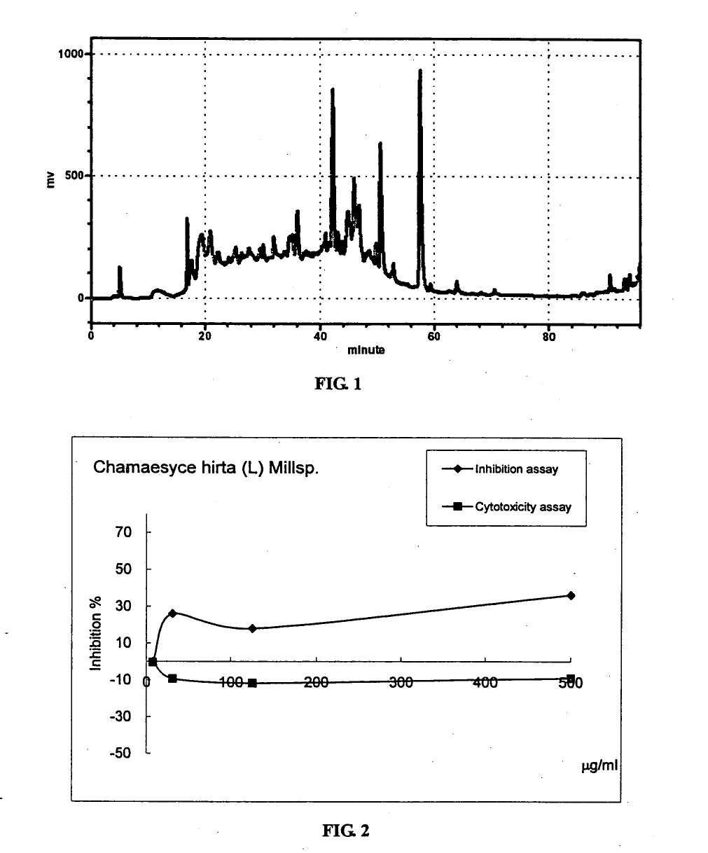

A compound having TNFα inhibitor activity was found in the methanolic extract of Chamaesyce hirta (L) Millsp. by using the procedures described above. In FIG. 1, a chromatogram of the crude methanolic extract of Chamaesyce hirta (L) Millsp. is shown. The crude methanolic extract of Chamaesyce hirta (L) Millsp. was fractionalized on a TSK Gel ODS 80™(TOSOH) reverse phase column. The particle size of the gel in this column was 5 μm, and the column size was 250×4.6 mm. The mobile phase used was a mixture of H₂O (A buffer) and absolute ethanol (B buffer) at a flow rate of 0.75 mL/min. The column was sequentially eluted as follows: 0% B for the first 5 minutes; a linear gradient of 0˜15% B for 15 minutes; 15˜50% B for 60 minutes; 50˜100% B for 10 minutes and 100% B for 6 minutes. The detection was performed at a wavelength of 280 nm with a detection sensitivity of 0.01 AUFS.

3. L929 Cellular Assay

Cell Culture

L929 cells were cultured in Eagle's Minimal Essential Medium (MEM) containing 10% equine serum, 1% P/S and 1% non-essential amino acid. Confluent L929 cells were washed with 2 ml PBS (phosphate-buffered saline) solution and then trypsinized with 1 ml 1×trypsin, followed by resuspending in complete medium. Two hundred microliter of cell suspension was aspirated for cell density counting. The remainder was centrifuged at 1500 rpm for 5 min. The supernatant was removed and the complete medium was added to dilute cells at a concentration of 1.5×10⁵ cells/ml. Add 100 μl of cell suspension to each well in 96-well flat-bottomed microtitre plates and incubated for 24 hrs in 5% CO₂ atmosphere at 37° C. incubator.

TNFα Activity Assay

Crude herbal extracts were resuspended in 1×PBS and sterilized with 0.22 μm filters. Varying concentrations of herbal extract were incubated for 1 hr with equal volume of commercial TNFα 0.2 ng/ml. Before the end of the 1 hr pre-incubation, removing the medium from the 24 hr incubated 96-well plate, and added a 50 μl fresh medium containing 4 μg/ml of Actinomycin D into the 96-well plate. Transferred the 50 μl of pre-incubated mixture of herbal extraction and TNFα to the 96-well plate with the medium containing Act D to give the final concentration of Act D (2 μg/ml), TNFα(0.1 ng/ml). The mixture of Act D (2 μg/ml) and TNFα (0.1 ng/ml) were added as positive control and Act D 2 μg/ml only was used as negative control. Alter gently shaking for 24 hrs in 5% CO₂ atmosphere at 37° C. incubator.

Cytotoxicity

The same samples as those for TNFα activity assay were added to the 96-well plate with the medium containing Act D to give the final concentration of Act D 2 μg/ml. Mixed well by gently shaking and then incubated for 24 hrs in 5% CO₂ atmosphere at 37° C. incubator. 50 μl XTT mixture (XTT−1: XTT−2=50:1) was added to each well, and incubated in a CO₂ incubator for 4 hrs. Read with ELISA (enzyme-linked immunosorbent assay) reader at O.D (optical density) 490/630 nm.
Calculation of the TNFα Activity Inhibition and Cytotoxicity

$$TNF α Inhibition \% = \frac{O . D {.}_{dilut + TNF + Act .} - O . D {.}_{TNFa + Act}}{O . D {.}_{Act only} - O . D {.}_{TNFa + Act}} \times 100 \%$$

$$Cytotoxicity \% = \frac{O . D {.}_{dilut . + ActD}}{O . D {.}_{ActD only}} \times 100 \%$$

4. Quercitrin and Myricitrin Identification
(1) Thin-Layer Chromatography

For TLC experiment, precoated plates of silica gel 60F₂₅₄ (E. Merck) were used and spotting was done with capillary tubes. The plates were scanned on a UV observed box (Gamag). The solvent system was chloroform:methanol:ethyl acetate/MeOH=20/1.5 for pure quercitrin and ethyl acetate/MeOH=6/1 for pure myricitrin. TLC of the isolated quercitrin and myricitrin showed a single spot with its R_{f} value 0.63 and 0.6 in this solvent system.

(2) LC/MS Spectrum

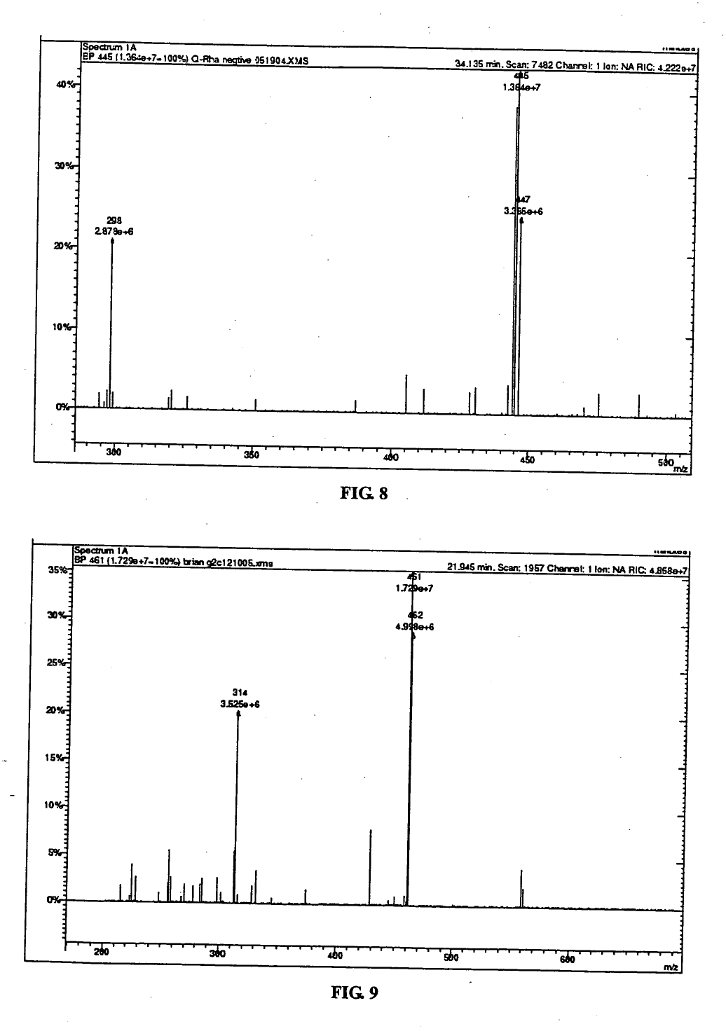

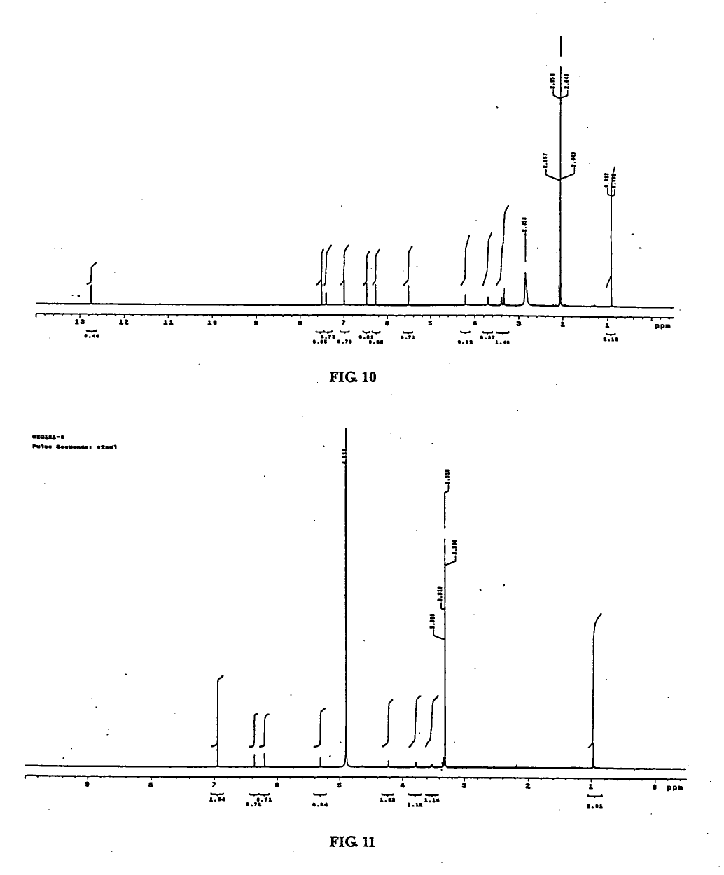

The atmospheric pressure ionization with ESI mass spectrum of molecular ions was obtained on a LC/MS (Varian). The mobile phase was water/EtOH. Quercitrin Mass: 445 (M+H)⁺ (FIG. 8), myricitrin 461 (M+H)⁺ (FIG. 9).

(3) HPLC Spectrum

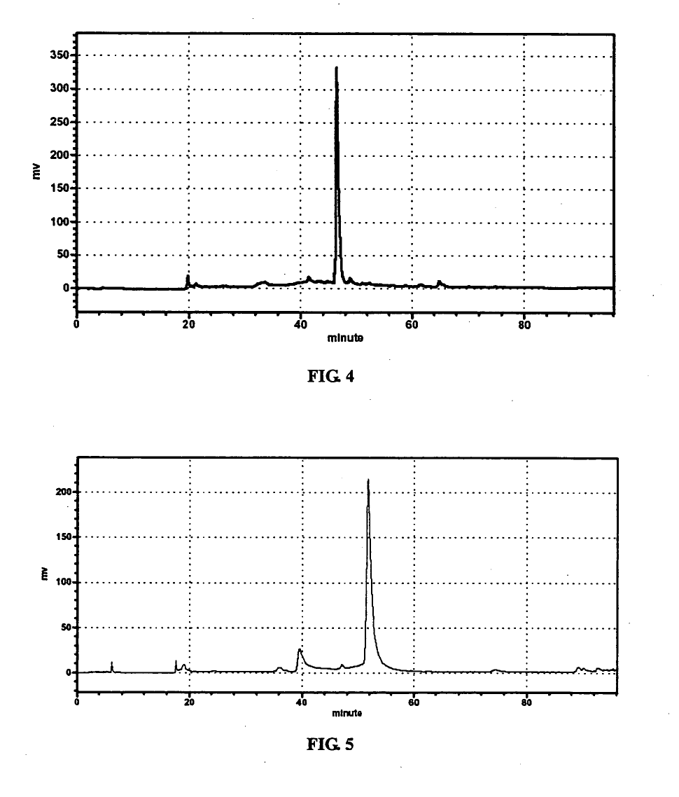

The HPLC spectra of quercitrin and myricitrin were obtained. The reference standard was obtained by TSK Gel ODS 80™ (5 μm) TOSOH reverse phase column (4.6×250 mm) using a Shimadu HPLC system with a mobile phase containing ethanol and water. The HPLC analysis of the quercitrin gave a single peak with retention time of 46.3 min (FIG. 4), and retention time of myricitrin was 51.8 min (FIG. 5). The following HPLC condition should be used when carrying out this analysis:

| Gradient Time (min) | B buffer (EtOH) % |
|---|---|
| 0˜5 | 0 |
| 5˜20 | 0˜15 |
| 20˜80 | 15˜50 |
| 80˜90 | 50˜100 |
| 90˜96 | 100 |

A buffer: H₂O

Flow Rate: 0.75 mL/min

Detection Wavelength: 280 nm

Injection volume: 100 μL

(4) ¹H-NMR Spectrum

The ¹H-NMR spectrum of quercitrin is shown in FIG. 10. ¹H-NMR (600 MHz, Acetone-d₆) δ0.91 (3H, d, J=6.0 Hz, Me rhamnose), 3.31-4.20 (4H, m, sugar protons), 5.52 (1H, d, J=1.2 Hz, H-1″), 6.26 (1H, d, J=1.8 Hz, H-6), 6.47 (1H, d, J=1.8 Hz, H-8), 6.99 (1H, d, J=7.8 Hz, H-5′), 7.40 (1H, dd, J=2.4, 7.8 Hz, H-6′), 7.50 (1H, d, J=2.4 Hz, H-2′).

The ¹H-NMR spectrum of myricitrin is shown in FIG. 11. ¹H NMR (600 MHz, CD₃OD) δ 0.96 (3H, d, J=6.0 Hz, Me rhamnose), 3.31-4.20 (4H, m, sugar protons), 5.31 (1H, d, J=1.2 Hz, H-1″), 6.26 (1H, d, J=1.8 Hz, H-6), 6.36 (1H, d, J=2.4 Hz, H-8), 6.95 (2H, s, H-2′ and H-6′).

5. Anti-Inflammatory Effect of Myricitrin and Quercetin-3-D-glucoside on Rats With Collogen-Induced Arthritis

SD rats of SPF grade were supplied from BioLasco. Prior to performing the study, the animals were accommodated for 4 days after being received. Weighing, blood sampling, measuring the paw volumes and other related records for each animal were established. The rats were immunized and boosted with bovine collagen II-EFA (Incomplete Freund's Adjuvant, from Sigma) to induce arthritis (CIA). The CIA rats were grouped into 6 groups and daily injected with the drug candidates (myricitrin and quercetin-3-D-glucoside respectively). Dexamethasone (0.2 mg) was used as a positive control and 5% ethanol as a negative control. Treatment period was 7 days. Body weight and paw volumes were measured and blood sampling were collected at day 0, 3, 6, 10 and 14.

Six days after the final dosing, all the animals were sacrificed. The affected hind limbs were removed for histological assessment. The parameters of body weights and paw volumes were measured and compared for before, during and after treatment with drug candidates.

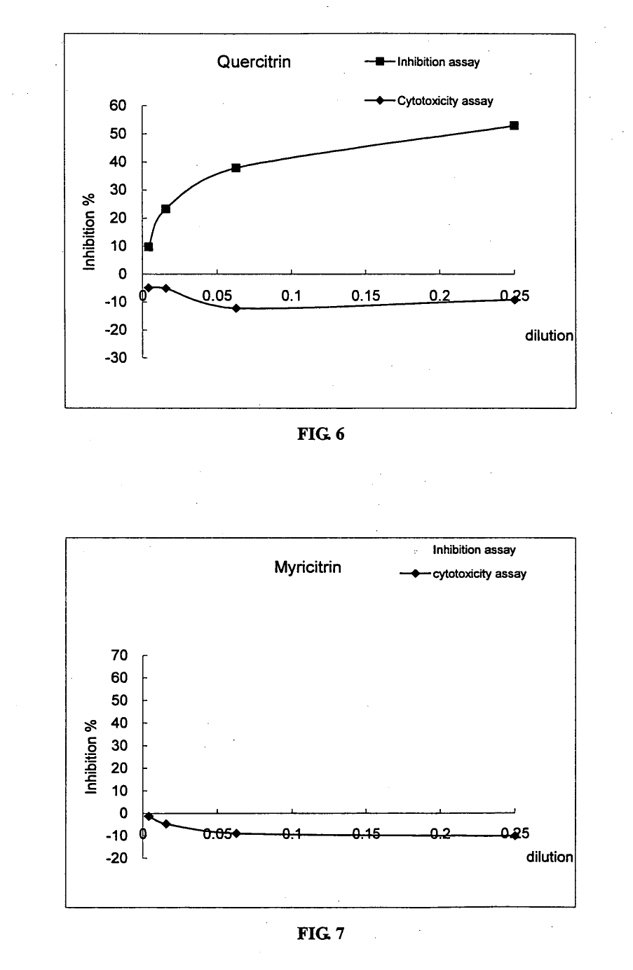

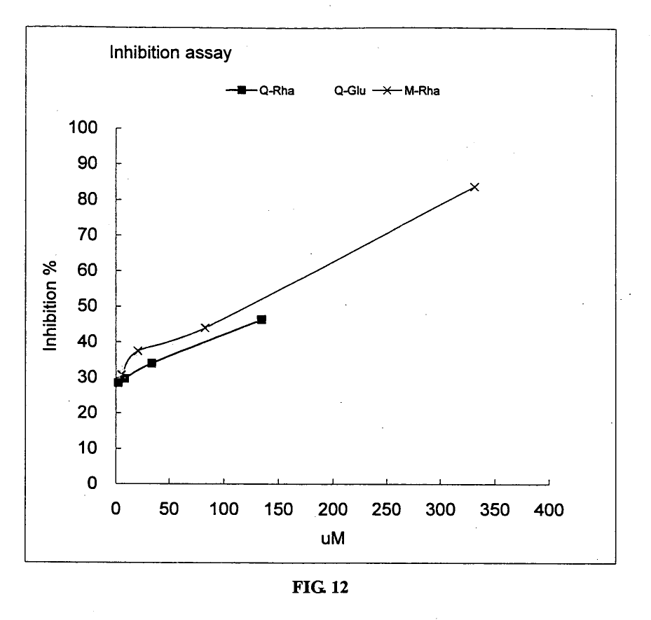

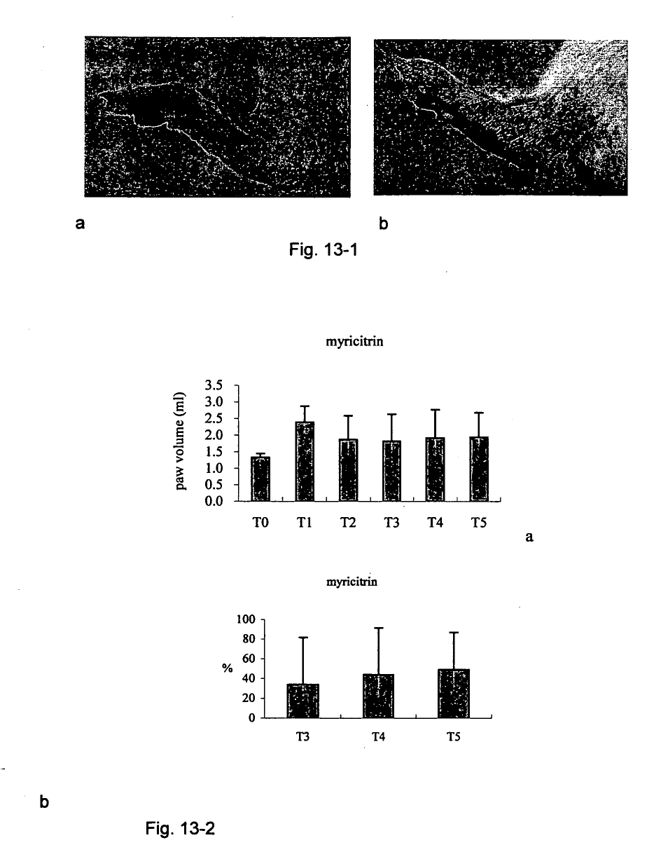

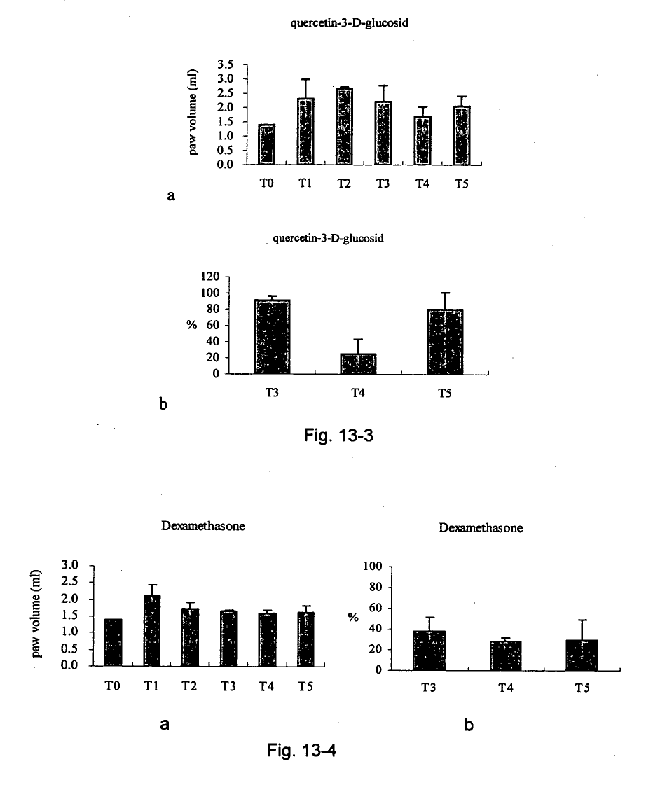

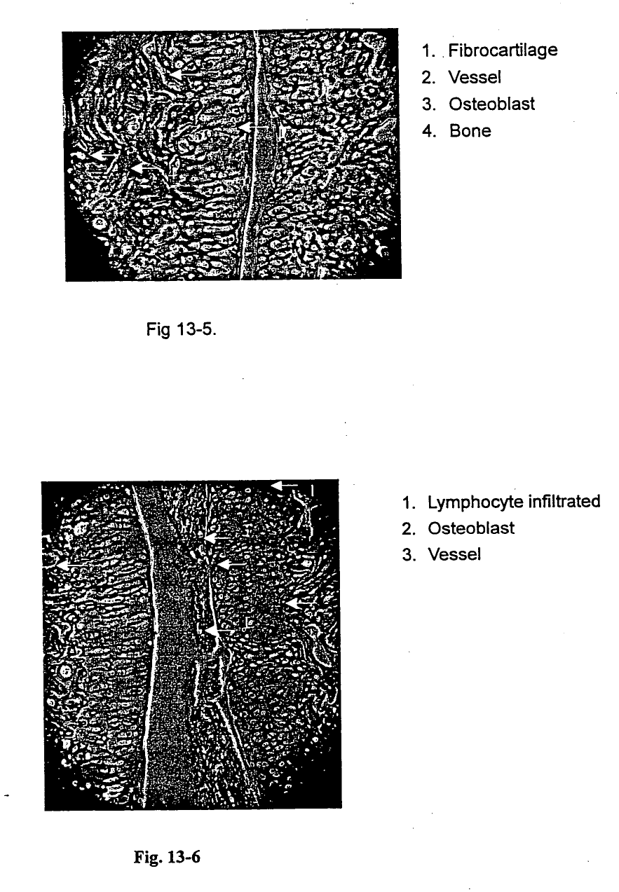

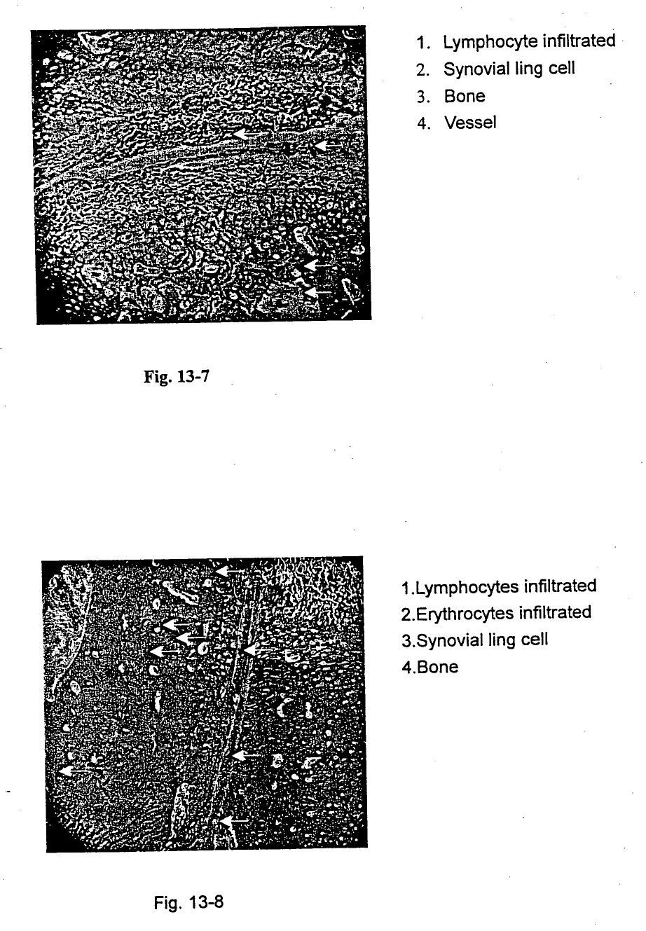

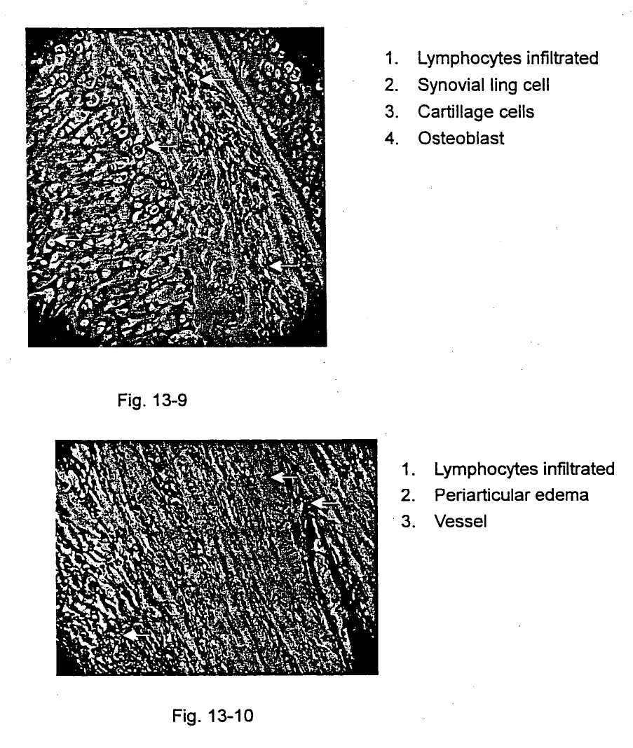

Collagen-induced arthritis was found on day 9th after boostering, the volumes of hind paw swelled 2-2.5 times that of normal hind paws. (See FIG. 13-1 in which FIG. 13-1a shows hind paw before CII-IFA injection. FIG. 13-1b shows hind paw with collagen-induced arthritis. Swelling and erythema appeared.) The group treated with myricitrin showing decreased percentage, 65.98%, of edema volumes for hind paws after continual treatment for 6 days. On the 3^{rd} day and 7^{th} day after treatment stopped, the decreased percentage of edema were 55.95% and 50.93% for myricitrin. (See FIG. 13-2, in which FIG. 13-2a shows volumes of left hind paw for group myricitrin. The volume of T0 is before injected CII-IFA, T1 is before treatment, T3 is day 6th of treatment, T4 and T5 are day 3rd and day 7th after administered. FIG. 13-2b shows different time points of edema percentage comparison with non-treatment volume of paw. T3 is 1−(T3−T1/T1−T0)%, T4 is 1−(T1-T4/T1−T0)% and T5 is 1−(T1-T5/T1−T0)%.). In the group treated with quercetin-3-D-glucoside, it appeared slight decrease percentage of edema volume in the treatment period (8.59%) in comparison with non-treatment. After stop administer day 3rd the decrease percentage was down to 24.93% and increase to 80.47% on day 7th. (See FIG. 13-3, in which FIG. 13-3a shows volumes of left hind paw for group quercetin-3-D-glucoside. The volume of T0 is before injected CII-IFA, T1 is before treatment, T3 is day 6th of treatment, T4 and T5 are day 3rd and day 7th after administered. FIG. 13-3b shows different time points of edema percentage compared with non-treatment volume of paw. T3 is 1−(T3−T1/T1−T0)%, T4 is 1−(T1-T4/T1−T0)% and T5 is 1−(T1-T5/T1−T0)%.) While the group treated with dexamethasone was 28.21% on the 3^{rd} day and 29.97% on the 7^{th} day in decreased percentage of edema. (See FIG. 13-4, in which FIG. 13-4a shows volumes of left hind paw for group dexamethasone. The volume of T0 is before injected CII-IFA, T1 is before treatment, T3 is day 6th of treatment, T4 and T5 are day 3rd and day 7th after administered. FIG. 13-4b shows different time points of edema percentage compared with non-treatment volume of paw. T3 is 1−(T3−T1/T1−T0)%, T4 is 1−(T1-T4/T1−T0)% and T5 is 1−(T1-T5/T1−T0)%.) Histopathological changes with loose connective tissues, lymphocytes infiltration around joint, periarticular edema and proliferation of synovial ling cells were observed in all arthritis samples (FIG. 13-6 to FIG. 13-10) but not in normal samples (FIG. 13-5). FIG. 13-5 shows a normal histological slice of joint of non-immune with collagen II. FIG. 13-6 shows a histopathological slice of rats with CIA and treated (IP) with myricitrin, in which proliferation of cell and infiltration of lymphocytes could be observed. FIG. 13-7 shows a histopathological slice of rats with CIA and treated (IP) with quercetin-3-D-glucoside, in which proliferation of synovial ling cell and infiltration of lymphocytes was shown. FIG. 13-8 shows a histopathological slice of rats with CIA and treated (IP) with dexamethasone. Proliferation of synovial ling cell and infiltration of erythrocytes and some lymphocytes could be observed. FIG. 13-9 shows a histopathological slice of rats with CIA and treated (IP) with 5% ethanol. Proliferation of synovial ling cell and infiltration of lymphocytes could be observed. FIG. 13-10 shows a histopathological slice of rats with CIA treated with dexamethasone. Periarticular edema and infiltration of lymphocytes were observed.

1. A pharmaceutical composition for antagonizing or inhibiting TNFα in a mammal, including human, comprising an amount of the compound of formula (I) 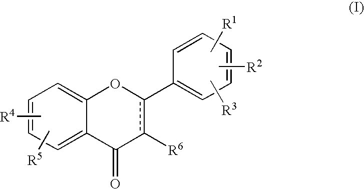
in which R¹, R², R³, R⁴ and R⁵ independently represent hydrogen, hydroxy or an ester group; R⁶ represents hydrogen, hydroxy, an ester group or an O-glycoside group such as O-rhamnose, O-glucoside, O-retinoside or O-xyloside; and [symbol] represents a single bond or a double bond; or the pharmaceutically acceptable salt thereof effective in antagonizing or inhibiting TNFα and a pharmaceutically acceptable carrier.

2. The pharmaceutical composition of claim 1, wherein the compound of formula (I) is myricitrin, quercitrin or quercetin-3-D-glucoside.

3. A pharmaceutical composition for treating a disease or condition for which a TNFα antagonist or inhibitor is indicated in a mammal, including human, comprising an amount of the compound of formula (I) or the pharmaceutically acceptable salt thereof as defined in claim 1 effective in antagonizing or inhibiting TNFα and a pharmaceutically acceptable carrier.

4. The pharmaceutical composition of claim 3, wherein the compound of formula (I) is myricitrin, quercitrin or quercetin-3-D-glucoside.

5. The pharmaceutical composition of claim 3, wherein the disease or condition is rheumatoid arthritis, Crohn's disease, plaque sclerosis, septic shock, cancer or cachexia associated with an immunodeficiency.

6. A method for antagonizing or inhibiting TNFα in a mammal, including human, comprising administering to said mammal an amount of the compound of formula (I) or the pharmaceutically acceptable salt thereof effective in antagonizing or inhibiting TNFα.

7. The method of claim 5, wherein the compound of formula (I) is myricitrin, quercitrin or quercetin-3-D-glucoside.

8. A method for treating a disease or condition for which a TNFα antagonist or inhibitor is indicated in a mammal, including human, comprising administering to said mammal an amount of the compound of formula (I) or the pharmaceutically acceptable salt thereof effective in antagonizing or inhibiting TNFα.

9. The method of claim 8, wherein the compound of formula (I) is myricitrin, quercitrin or quercetin-3-D-glucoside.

10. The method of claim 8, wherein the disease or condition is rheumatoid arthritis, Crohn's disease, plaque sclerosis, septic shock, cancer or cachexia associated with an immunodeficiency.

11. A compound of formula (I) 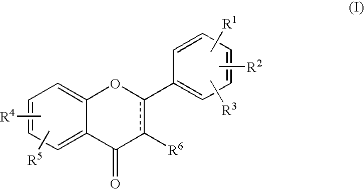
in which R¹, R², R³, R⁴ and R⁵ independently represent hydrogen, hydroxy or an ester group; R⁶ represents hydrogen, hydroxy, an ester group or an O-glycoside group such as O-rhamnose, O-glucoside, O-retinoside or O-xyloside; and [symbol] represents a single bond or a double bond, or a pharmaceutically acceptable salt thereof, with the proviso that the compound is not myricitrin, quercitrin or quercetin-3-D-glucoside.

12. The pharmaceutical composition of claim 1, wherein the compound of formula (I) is not myricitrin, quercitrin or quercetin-3-D-glucoside.

13. The pharmaceutical composition of claim 3, wherein the compound of formula (I) is not myricitrin, quercitrin or quercetin-3-D-glucoside.

14. The pharmaceutical composition of claim 4, wherein the disease or condition is rheumatoid arthritis, Crohn's disease, plaque sclerosis, septic shock, cancer or cachexia associated with an immunodeficiency.

15. The pharmaceutical composition of claim 13, wherein the disease or condition is rheumatoid arthritis, Crohn's disease, plaque sclerosis, septic shock, cancer or cachexia associated with an immunodeficiency.
